(19) United States
(12) Patent Application Publication
(10) Pub. No.: US 2006/0264774 A1
(43) Pub. Date: Nov. 23, 2006

(54) Neurologically Controlled Access to an Electronic Information Resource

(75) Inventor: Louis B. Rosenberg, Pismo Beach, CA (US)

Correspondence Address: LAW OFFICE OF PHILIP A STEINER, 846 HIGUERA STREET, SUITE 4, SAN LUIS OBISPO, CA, 93401, US

(73) Assignee: OUTLAND RESEARCH, LLC, Pismo Beach, CA (US)

(21) Appl. No.: 11/464,787

(22) Filed: Aug. 15, 2006

Related U.S. Application Data
(60) Provisional application No. 60/711,535, filed on Aug. 25, 2005.

(51) Int. Cl. A61B 5/04

(52) U.S. Cl. 600/544

(57) ABSTRACT

A method and system for an embeddable electronic information resource which may be cognitively enabled by a human host using a neurological electronic interface. In an embodiment, the electronic information resource is provided as an RFID device or an ISO-14443 compatible device. The various embodiments allow the human host to selectively enable and/or disable the embeddable electronic information resource through thought-commands issued to the neurological electronic interface, thus providing an affirmative access control of the information contained in the electronic information resource. Embodiments are provided which alerts the human host through direct neural feedback, about the state and/or status of accesses and/or attempted accesses to the electronic information resource, and/or about the result of an authentication performed by a remote device that has just scanned the electronic information resource.

### CROSS-REFERENCE TO RELATED APPLICATIONS

This application claims benefit and priority under 35 U.S.C. § 119(e) from U.S. provisional patent application Ser. No. 60/711,535 filed Aug. 5, 2005 to the instant inventor and a common assignee; the aforementioned provisional application Ser. No. 60/711,535 is hereby incorporated by reference in its entirety as if fully set forth herein.

### FEDERALLY SPONSORED RESEARCH AND DEVELOPMENT

Not Applicable

### REFERENCE TO A MICROFICHE APPENDIX

Not Applicable

### RELEVANT FIELD

A data processing arrangement is described for controlling access to host specific information contained in a remotely interrogated device. More specifically, various exemplary embodiments provide a system and method for neurologically controlling access to an electronic information resource.

### BACKGROUND

In recent years, miniaturized implantable electronic devices have been developed that may hold private information about its host. As disclosed in U.S. patent application 20030195523, published Oct. 16, 2003 to Futsz, and hereby incorporated by reference, describes such devices, once they are implanted, are generally dormant until interrogated by an external scanner, at which time they transpond with an encoded radio frequency signal that can be read remotely by the scanner. In operation, an implanted device may provide hospitals and emergency workers with critical medical information about a patient who has such an implanted chip. Similarly, an implantable chip may provide security information for verification of a host in accessing secure locations, secure equipment, and/or secure information.

Such information may further include host identification and password information. Similarly an implantable device may be used to identify a host for banking transactions, file transfers, computer login, communication connections, and demographic data collection. Unfortunately, implantable devices known in the relevant art are vulnerable to unknown and/or unwanted interrogation. For example a host walking past a hidden scanner may have his private information covertly scanned and accessed without permission.

Therefore, a mechanism by which a host can selectively enable and/or disable an implantable device such that only when purposefully enabled and/or authorized by the host may an external scanner access some or all of the host's private information and without requiring any overt movements by the host.

### SUMMARY

Various embodiments are described for an implantable and remotely accessible electronic information resource, such as an RFID tag or contactless smartcard chip loaded with host specific data, which is interfaced with a host's nervous system such that the host can enable and/or disable access to the host specific data through cognitive neurological activity. The various embodiments detect the neurological activity of one or more neurons associated with the host's central or peripheral nervous system and using that activity to enable and/or disable the access to the electronic information resource.

In an exemplary systematic embodiment, a system for neurologically controlling access to an electronic information resource is provided. This exemplary systematic embodiment comprises; a neurological electronic interface operatively coupled to a neurological sensor and an electronic information resource.

The neurological electronic interface includes a neurological processing unit programmed to determine whether bioelectrical signals received from the neurological sensor are indicative of a cognitive selection state; and permissively allows access to the electronic information resource in dependence on the determined cognitive selection state.

In addition, the neurological sensor is configured to transmit the bioelectrical signals generated by a nervous tissue in which it is in bioelectrical contact, to the neurological electronic interface and the electronic information resource including information permissively available for remote interrogation in dependence on the determined cognitive selection state.

In a first related exemplary systematic embodiment, the electronic information resource is one of an RFID device and an ISO-14443 compliant device.

In a second related exemplary systematic embodiment, the bioelectrical signals are generated by the nervous tissue associated with one or more of a peripheral nervous system and central nervous system.

In a third related exemplary systematic embodiment, one or more of the neurological electronic interface, the electronic information resource and the neurological sensor is in bioelectrical contact at least subcutaneously.

In a fourth related exemplary systematic embodiment, the neurological processing unit is further programmed to generate a neural feedback signal in dependence on the electronic information resource being remotely interrogated.

In a fifth related exemplary systematic embodiment, the neurological processing unit is further programmed to generate a neural feedback signal in dependence on the determined cognitive selection state.

In a sixth related exemplary systematic embodiment, the neurological processing unit is further programmed to generate at least one neural feedback signal in dependence on the electronic information resource being remotely interrogated and an access state.

In a seventh related exemplary systematic embodiment, the at least one neural feedback signal comprises a plurality of perceptionally distinct neural feedback signals generated in dependence on the access state and applied to the nervous tissue in which the neurological sensor is in bioelectrical contact.

In an eighth related exemplary systematic embodiment, the access state is one of allowed and rejected.

In a ninth related exemplary systematic embodiment, the neurological processing unit is further programmed to provide bi-directional communications between the nervous tissue and the electronic information resource.

In a tenth related exemplary systematic embodiment, a second neurological electronic interface is operatively coupled to another neurological sensor and the electronic information resource and configured to generate the neural feedback signals in cooperation with the neurological electronic interface.

In an exemplary methodic embodiment, a method for neurologically controlling access to an electronic information resource is provided. This exemplary methodic embodiment comprising; providing a neurological electronic interface programmed to determine whether bioelectrical signals received from a neurological sensor are indicative of a cognitive selection state and permissively allow access to an electronic information resource in dependence on the determined cognitive selection state;

providing the electronic information resource configured to operably couple to the neurological electronic interface; where the electronic information resource includes information permissively available for remote interrogation in dependence on the determined cognitive selection state; and,

providing the neurological sensor configured to operably couple to the neurological electronic interface; where the neurological sensor is configured to transmit the bioelectrical signals generated by a nervous tissue in which it is to be in bioelectrical contact to the neurological electronic interface.

In a first related methodic embodiment, the electronic information resource is one of an RFID device and an ISO-14443 compliant device.

In a second related methodic embodiment, the bioelectrical signals are generated by the nervous tissue associated with one of; a peripheral nervous system and a central nervous system when the neurological sensor is in bioelectrical contact therewith.

In a third related methodic embodiment, one of; the neurological electronic interface, the electronic information resource, the neurological sensor and any combination thereof is embeddable at least subcutaneously in a human host.

In a fourth related methodic embodiment, the neurological electronic interface is further programmed to generate a neural feedback signal in dependence on the electronic information resource being remotely interrogated.

In a fifth related methodic embodiment, the neurological electronic interface is further programmed to generate a neural feedback signal in dependence on the determined cognitive selection state.

In a sixth related methodic embodiment, the neurological electronic interface is further programmed to generate at least one neural feedback signal in dependence on an access state associated with the electronic information resource.

In a seventh related methodic embodiment, the access state is one of allowed and rejected.

In an eighth related methodic embodiment, the neurological electronic interface is further programmed to provide bi-directional communication between the nervous tissue and the electronic information resource.

The various exemplary systematic and methodic embodiments described above are provided in related numeric embodiments for convenience only. No limitation to the various exemplary embodiments disclosed is intended.

### BRIEF DESCRIPTION OF THE DRAWING

The features and advantages will become apparent from the following detailed description when considered in conjunction with the accompanying drawings. Where possible, the same reference numerals and characters are used to denote like features, elements, components or portions. Optional components or feature are generally shown in dashed or dotted lines. It is intended that changes and modifications may be made to the described exemplary embodiments without departing from the true scope and spirit of the subject invention.

FIG. 1—provides an exemplary general block diagram of a neural interface device coupled to a neurological sensor and a remotely interrogated electronic information resource.

FIG. 1A—provides an exemplary general block diagram of the remotely interrogated electronic information resource.

FIG. 2—provides exemplary implementation embodiments of the remotely interrogated electronic information resource in bioelectrical contact with a host.

FIG. 3—provides an exemplary process flow chart of the various exemplary embodiments.

### DETAILED DESCRIPTION

A plurality of mechanisms is available in the relevant art which may be used to cognitively control an electronic device. For example, the Brain Gate™ product supplied by Cyberkinetics (www.cyberkineticsinc.com) provides an array of electrodes and processing electronics which may be used to perform the required neurological electronic interface functions as described herein. The technology from Cyberkinetics is described in detail in U.S. patent applications 20040082875 published Apr. 29, 2004, 20040249302 published Dec. 9, 2004 and 20050113744 published May 26, 2005 all of which are hereby incorporated by reference. As is discussed in U.S. patent applications 20040249302 and 20050113744, the Cyberkinetics' technology allows for the creation of direct, reliable and bi-directional interfaces between the brain, nervous system and an electronic device.

Other devices and/or technologies are known in the relevant art of neural electronics which may be used separately or in combination with the technology from Cyberkinetics as described above. For example, Dr. Kensall Wise of University of Michigan in Ann Arbor, has developed an electronic probe that can be implanted deep into brain tissue and used send an receive information between the brain and electronic devices.

In another example, Dr. Eve Marder at Brandeis University has developed a system called the “Dynamic Clamp,” that enables communication between neurons and electronic devices. Electrical impulses are transmitted to a computer through probes inserted into a neuron. The neuron reacts as if it were communicating with another neuron, rather than a computer.

In another example, scientists from Emory University have implanted a chip in the brain of a paralyzed stroke victim that allows the victim to use his brainpower to move a cursor across a computer screen. Emory University neural scientist Philip R. Kennedy, M.D., and Emory neural surgeon Roy E. Bakay, M.D., have developed an electrode brain implant that is allowing speech-impaired patients to communicate through a computer.

In yet another example, Douglas J. Weber, PhD, at the University of Alberta, Edmonton, Canada, has developed an implantable microelectrode array that can inserted into the dorsal root ganglion of the spinal chord as a less invasive way to interface motor and/or sensory neurons with electronic devices.

In a final example, Jonathan Wolpaw, M.D., and Dennis McFarland, PhD of Department of Health's Wadsworth Center laboratories have developed a non-invasive system that collects neurological signals upon the surface of a person's skin, for example the scalp, or other dermal surfaces containing peripheral nervous tissue using surface electrodes. The collected signals are used to control electronic devices such as cursor upon a computer screen.

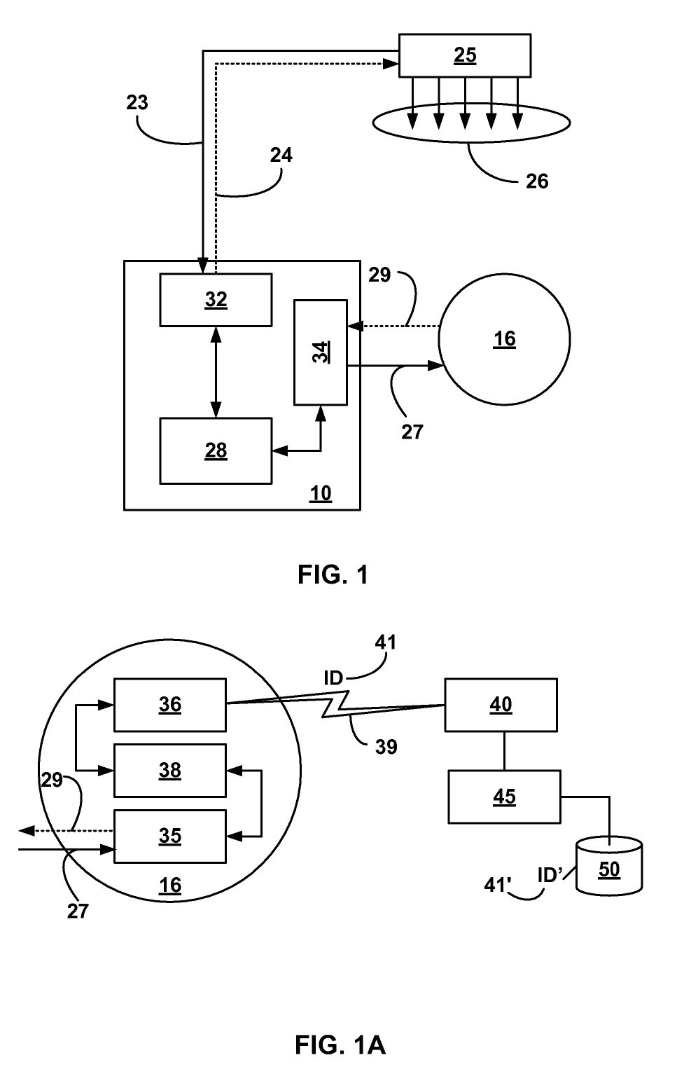

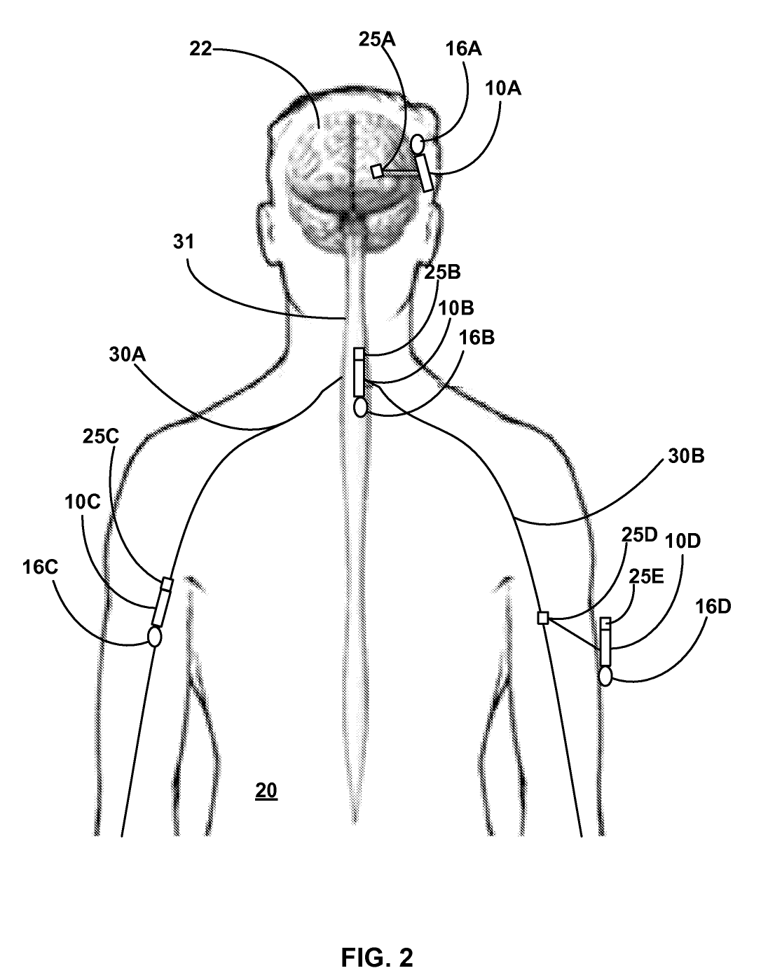

Referring to FIG. 1 an exemplary block diagram of an embodiment where a neurological sensor 25 is in bioelectrical contact with and/or embedded within nervous tissue 26 (FIG. 2) is provided. The nervous tissue as described herein includes brain tissue 22, central nervous system tissue 24 (FIG. 2) and/or peripheral nervous tissue 30A-C (FIG. 2.)

The neurological sensor 25 provides bioelectrical signals 23 to a neurological interface device 10. The neurological interface device 10 may include a signal conditioning circuit 32, for example, a preamplifier and/or an analog to digital converter to provide detected bioelectrical signals 23 in a usable form to a processor 28 associated with the neurological interface device 10. To process the detected bioelectrical signals, the processor 28 may include embedded table of values in a ROM or EEPROM (not shown), such as a look-up table of trigger points and/or transfer function variables or coefficients. The stored table values are then used to provide a cognitive electronic signal 27 to an electronic information resource 16 in response to particular detected bioelectrical signal. The stored table values may be customized for or by the individual host 20.

The processor 28 may also be programmed to conduct adaptive processing of the received bioelectrical signals 23 by changing one or more parameters of the system to achieve or improve performance. Examples of adaptive processing include, but are not limited to, changing a parameter during a system calibration, changing a method of encoding neural signal information, changing the type, subset, or amount of neural signal information that is processed, or changing a method of decoding neural signal information. Changing an encoding method may include changing neural spike sorting methodology, calculations, thresholds, or pattern recognition. Changing a decoding methodology may include changing variables, coefficients, algorithms, and/or filter selections.

In a related embodiment, the signal conditioning circuit 32 provides feedback signals 24 to the nervous tissue 26 via the neurological sensor 25. The neurological sensor 25 may include a plurality of electrodes for detecting bioelectrical signals or impulses transmitted between the neurons. In an embodiment, the neurological sensor 25 may be inserted into a host's cerebral cortex 22, or in any location of the host's brain allowing for the detection of bioelectrical signals or impulses.

In another embodiment, an electronic information resource interface 34 is provided which allows the processor 28 to communicate 27 with the electronic information resource 16. As is discussed in U.S. Pat. No. 5,963,144 which is hereby incorporated by reference, the electronic information resource 16 may be constructed such that an antenna associated with the electronic information resource 16 is disconnected from the balance of the electronic information resource 16 in response to a digital logic command.

Such a digital logic command may be produced by the electronic information resource interface 34 such that a host 20 can selectively enable and/or disable remote access to the electronic information resource 16 in response to his or her cognitive thought processes.

In an embodiment, the electronic information resource interface 34 may receive signals 29 generated by the electronic information resource 16. The electronic information resource 16 may be directly incorporated into the neurological interface device 10 or connected by a wireless communications link.

Additionally, suitable devices for supplying electrical power to the various electronic modules known in the relevant art may be employed (Not shown.) For example, the system may include one or more power supplies, such as batteries and/or bioelectrical generators. The power supply may be recharged (e.g., via inductive coupling) or may need to be replaced when the power is exhausted. The system may also include power supply means that draws power from RF energy produced by an external scanner.

The neurological electronic interface 10 may receive neurological signals using any suitable invasive or noninvasive neurological sensor 25 and may produce electronic signals in a variety of forms in response to the sensed neurological signals. For instance, the neurological sensor 25 may include noninvasive or substantially noninvasive sensors, such as one or more multi-channel electroencephalogram (EEG) sensors placed on or within the surface of the host's skin. The neurological sensor 25 may also be invasive sensor, such as an implanted electrode, set of electrodes, and/or an array of electrodes that detects neurological signals in the form of neural spikes, local field potentials (LFPs), or electrocortigram signals (EcoGs).

For example, U.S. Pat. No. 6,171,239 to Humphrey and entitled “Systems, Methods, and Devices for Controlling External Devices By Signals Derived Directly From the Nervous System,” and U.S. Pat. No. 5,215,088 to Normann, et al., entitled “Three-Dimensional Electrode Device,” each patent reference discloses electrode arrays suitable for use in the various embodiments described. The aforementioned patent references to Humphrey and Normann, et al. are hereby incorporated by reference. One skilled in the art will appreciate that other sensor arrays or probes capable of detecting neurological signals generated by the host 20 may be used in the various embodiments described herein.

In an embodiment, the neurological interface device 10 may be configured to provide the host 20 with direct neural feedback 24 that informs the host 20 by way of a neural feedback signal 24 if and when certain events occur. In another embodiment, a plurality of unique neural feedback signals 24 are selectively imparted, each of said plurality of unique neural feedback signals 24 being imparted such that they inform the host 20 when each of a plurality of certain unique events occur.

For example, the host 20 may be provided with a unique neural feedback signal 24 that informs the host 20 that the electronic information resource 16 has successfully transferred data to a transceiver. Similarly, the neurological interface device 10 may be configured to provide the host 20 with another unique neural feedback signal 24 that informs the host 20 that the electronic information resource 16 has rejected an access attempt from an unauthorized or incompatible transceiver 40.

In another example, the neurological interface device 10 may be configured to provide the host 20 with yet another unique neural feedback signal 24 that informs the host 20 that the electronic information resource 16 is has received a signal from a transceiver 40.

In a another example, the neurological interface device 10 may be configured to provide the host 20 with yet another unique neural feedback signal 24 that informs the host 20 that the electronic information resource 16 has been accessed, has transferred data to a transceiver 40, and/or the data transfer has resulted in the host 20 being successfully authenticated, authorized, or otherwise granted access to a service, application, device, or location. Similarly, the neurological interface device 10 may be configured to provide the host 20 with yet another unique neural feedback signal 24 that informs the host 20 that the electronic information resource 16 has been accessed, has transferred data to a transceiver 40, and/or the data transfer has resulted in the host 20 being rejected or otherwise unsuccessful in an attempt at being authenticated, authorized, or otherwise granted access to a service, application, device, or location. In this way, the a host may be alerted by unique neural feedback signals 24 as to the success or failure of an authentication assessment made by a remote device that has accessed information, such as user ID and/or password information, from the electronic information resource 16.

In the various examples provided above, the neural feedback signal 24 may be a voltage or current modulated electronic signal produced by a power amplifier and imparted upon the nerve tissue 26 of the host 20 via the neurological sensor 25. In this arrangement, an amplifier produces the neural feedback signals 24 under the control of the processor 28 in response to the signals 29 received from the electronic information resource 16.

FIG. 1A depicts an exemplary embodiment of the electronic information resource 16. The electronic information resource 16 may be configured as a radio frequency identification (RFID) tag, for example, devices compliant with the International Standards Organization (ISO) 15961 or a contactless smartcard chip which is compliant with the ISO-14443 series. One skilled in the art will appreciate that multiple international standards apply to each device configuration.

In either configuration, the electronic information resource 16 may include a complementary neurological interface 35 to allow communications with the electronic information resource interface 34 associated with the neurological interface device 10. The electronic information resource 16 generally includes a microprocessor 38 coupled to a radio frequency (RF) transponder 36. The transponder 36 receives properly encoded radio frequency signals which are used to both power the RFID device 16 and transpond information 39 to an external transceiver 40.

In the RFID tag embodiment, the transponded information 39 includes a unique identification code (ID) 41 which may be used to relationally retrieve information associated with a host 20 (FIG. 2) from a computer system 45. In this embodiment, the retrievable information is indexed by the unique ID 41, 41′ and is stored in a datastore 50 coupled to the computer system 45. The datastore 50 may be coupled to the computer system 45 directly or via a network (not shown.)

In the contactless smartcard chip embodiment, the electronic information resource 16 may provide additional information about the host 20 such as a personal information, identification information, medical information, authentication information, and/or financial information. This additional information may be cryptographically encoded as is known in the relevant art associated with contactless smartcards.

FIG. 2 provides a plurality of exemplary embodiments for the electronic information resource 16A-D. Depending on the particular embodiment, any or all of the electronics comprising the neural electronic interface 10A-D, neurological sensor 25A-D and electronic information resource 16A-D may be embedded with the body or cranial cavity of the host 20. In the embedded electronics embodiments, the neurological interface 10A-D may be used to detect signals from the host's brain 22, the host's spinal chord 24, and/or one or more of the host's peripheral nerves 30A,B. In an alternate embodiment, all of the electronics comprising the neural electronic interface 10A-D, neurological sensor 25A-D and electronic information resource 16A-D may be external to the body or cranial cavity of the host 20. In this alternate embodiment, the neurological sensor 25E is in bioelectrical contact with an enervated epidermal tissue.

In an embodiment, the host 20 has an electronic information resource 16A implanted within his or her skull cavity. A neurological sensor 25 is in contact with the brain 22 of the host 20 and is connected to the neurological interface device 10A by a wire link. The electronic information resource 16A may be connected to the neurological interface device 10A by a wire or wireless connection. In this embodiment, cognitive bioelectrical signals are detected by the neurological sensor 25A and processed by the neurological interface device 10A. If the host 20 desires to allow access to the embedded electronic information resource 16A, he or she consciously thinks about allowing access which is detected by the neurological sensor 25A and processed by the neurological interface device 10A. Access to the electronic information resource 16A is permitted only during the time period in which the host 20 is cognitively granting access. In an alternate embodiment, the host 20 only needs to cognitively grant access to the electronic information resource 16A for a brief instant.

Once access is cognitively granted by the host 20, a time delay may be invoked which allows access to the electronic information resource 16A for a preset time. The duration of the time delay is arbitrary and may be set for any reasonable amount of time necessary for the transceiver 40 to receive the information transponded to the transceiver 40. For example, a time delay of 5 to 10 seconds may be used in some embodiments to provide sufficient time for a remote transceiver 40 to gain access to the electronic information resource 16 and transfer data from it.

In another embodiment, the host 20 has an electronic information resource 16B implanted along the host's spinal chord 24. A neurological sensor 25B is in contact with the spinal cord 31 of the host 20 and is connected to the neurological interface device 10B by a wire or wireless connection. The electronic information resource 16B may be connected to the neurological interface device 10B analogously. In a third embodiment, the host 20 has an electronic information resource 16C implanted along a peripheral nerve 30A of the host 20. The neurological sensor 25C is in contact with the peripheral nerve 30A of the host 20 and is connected to the neurological interface device 10C by a wire or wireless connection. The electronic information resource 16C may be connected to the neurological interface device 10C analogously.

In a non-invasive fourth embodiment, the neurological sensor 25E is in contact with an enervated epidermal tissue of the host 20. The nervous tissue enervating the epidermal tissue is derived from one or more peripheral nerve branches 30B. In this embodiment, the neurological sensor 25E is connected to the neurological interface device 10D by a wire or wireless connection. The electronic information resource 16D may be connected to the neurological interface device 10D analogously by wired or wireless connections.

Operation of the various embodiments is substantially similar to that of the invasive cranial embodiment where cognitive bioelectrical signals are detected by the neurological sensors 25A-E and processed by the neurological interface device 10A-D. As was previously described, if the host 20 desires to allow access to the electronic information resource 16A-D, he or she consciously thinks about allowing access which is detected by the neurological sensor 25A-E and processed by the neurological interface device 10A-D.

Access to the electronic information resource 16A-D is permitted only during the time period in which the host 20 is cognitively granting access. Again as previously described, in an alternate embodiment, the host 20 only needs to cognitively grant access to the electronic information resource 16A-D for a brief instant and the processor provides access for a period thereafter. In some such embodiments the processor provides access until the host explicitly rejects access through the issuance of a subsequent cognitive command. In some such embodiments the subsequent cognitive command is specifically an access denial command issued by the host by consciously thinking about denying access.

One skilled in the art will appreciate that the neurological sensor 25A-E, neurological interface device 10A-D and/or electronic information resource 16A-D may be integrated into a single form factor or separated as necessary to meet a particular requirement.

It should be noted that the use of a plurality of separately placed neurological electronic interfaces 10A,B may be used in combination to detect the bioelectrical signals 23 generated by the host 20 to control the electronic information resource 16A accordingly and may provide neural feedback signals 24 in response to signals received from the electronic information resource 16A. For example, two neurological electronic interfaces 10A,B may be used as follows; the first neurological electronic interface 10A may be implanted in the host's brain 22 sends an electronic signal 27 to the electronic information resource 16A in response to detected bioelectrical signals 23 and a second neurological electronic interface 10B implanted along the spinal cord 31 of the host provides a neural feedback signal 24 upon the spinal cord tissue of in response to an electrical signal received from the electronic information resource 16A.

Such a configuration that employs separately placed neurological electronic interfaces 10A,B is beneficial for a number of reasons. One reason is that these arrangements act to reduce and/or eliminate cross-talk between electrodes that detect bioelectrical signals 23 generated by the host 20 and neural feedback signals 24 that stimulate neurological tissue. In other words, by detecting bioelectrical signals 23 using neurological sensors 25A at a first location with the host's body 20 and providing neural feedback signals 24 neurological tissue using neurological sensors 25B at a second location with the host's body 20, this configuration reduces the cross-talk between input and output signals in the bi-directional communication link between the electronic information resource 16A and the host's nervous system.

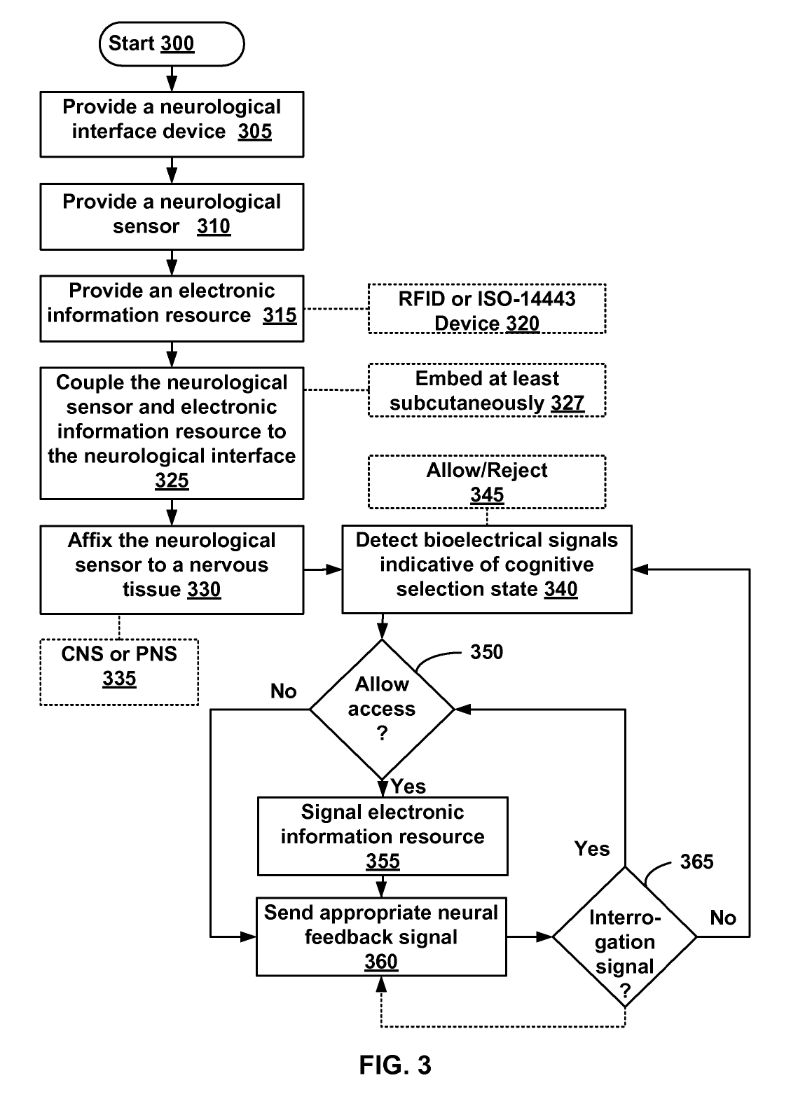

FIG. 3 provides an exemplary process flow chart of the various exemplary embodiments. The process is initiated 300 by providing; a neurological interface device 305, a neurological sensor 310 and an electronic information resource 315, for example an RFID device or an ISO-14443 compatible device 320. The neurological sensor and electronic information resource are then coupled to the neurological interface device 325.

In an embodiment, the assembled electronics may be embedded at least subcutaneously in a human host 327. In another embodiment, the electronic information resource and/or the neurological sensor may be integrated into the neurological interface device. As such, the latter and former steps may be eliminated as appropriate. The neurological sensor is then affixed to a nervous tissue 330 of a host, for example, the nervous tissue may be that of the central nervous system (CNS) or peripheral nervous system (PNS) 335. The peripheral nervous tissue may also enervate dermal tissues.

The neurological sensor is configured to detect bioelectrical signals indicative of a cognitive state selection 340, for example allow/reject access to the electronic information resource 345. If the host cognitively allows access to the electronic information resource 350, the electronic information resource is signaled which is then enabled to allow access by remote interrogation 355. In an embodiment, an appropriate neural feedback signal is sent to the neurological sensor alerting the host that the electronic information resource is enabled for interrogation 360.

In an embodiment, if the host cognitively rejects access to the electronic information resource 350, an appropriate neural feedback signal is sent to the neurological sensor alerting the host that the electronic information resource is disabled for interrogation 360.

In an embodiment, the electronic information resource is configured to signal the neurological interface device when an interrogation signal is detected. In this embodiment, if the electronic information resource detects an interrogation signal 365, the host may be notified by an appropriate neural feedback signal 360 followed by the host cognitively allowing or rejecting access to the electronic information resource 350 as described above. If an interrogation signal 365 is absent, the process continues in a loop where the neurological sensor detects bioelectrical signals indicative of cognitive state selection 340 as described above. In this way, a host may be neurologically alerted when a remote scanner attempts to access the embedded electronic information resource 16 within him or her. In response to this alert, the host may then mentally grant access and/or mentally reject access to the remote scanner by issuing an appropriate mental command (i.e. by thinking in such a way that a correct neural signal patterns is produced). Furthermore, a host may be informed as to the status of and/or result of the information access by the remote scanner through a neural feedback signal. In some such embodiments the host is informed as to the success or failure of an authentication process that is performed in response to the remote scanner access.

In this way a host may be informed through neural feedback if a successful authentication has occurred and thereby granted that host access to a service, application, device, or location. Where necessary, computer programs, algorithms and routines may be programmed in a high level language object oriented language, for example Java™ C++, C#, or Visual Basic™.

The various exemplary embodiments described herein are merely illustrative of the principles underlying an inventive concept. It is therefore contemplated that various modifications of the disclosed exemplary embodiments will, without departing from the spirit and scope of the various exemplary inventive embodiments will be apparent to persons of ordinary skill in the art. In particular, it is contemplated that functional implementation of the various exemplary embodiments described herein may be implemented equivalently in hardware, software, firmware, and/or other available functional components or building blocks. No specific limitation is intended to a particular method, system or process sequence. Other variations and exemplary embodiments are possible in light of above teachings, and it is not intended that this Detailed Description limit the scope of invention, but rather by the Claims following herein.

What is claimed:

1. A system for neurologically controlling access to an electronic information resource comprising:
a neurological electronic interface operatively coupled to a neurological sensor and an electronic information resource; the neurological electronic interface including;
a neurological processing unit programmed to determine whether bioelectrical signals received from the neurological sensor are indicative of a cognitive selection state; and, permissively allow access to the electronic information resource in dependence on the determined cognitive selection state;
the neurological sensor configured to transmit the bioelectrical signals generated by a nervous tissue in which it is in bioelectrical contact to the neurological electronic interface; and,
the electronic information resource including information permissively available for remote interrogation in dependence on the determined cognitive selection state.

2. The system according to claim 1 wherein the electronic information resource is one of; an RFID device and an ISO-14443 compliant device.

3. The system according to claim 1 wherein the bioelectrical signals are generated by the nervous tissue associated with one of; peripheral nervous system and central nervous system.

4. The system according to claim 1 wherein one of; the neurological electronic interface, the electronic information resource and the neurological sensor is in bioelectrical contact at least subcutaneously.

5. The system according to claim 1 wherein the neurological processing unit is further programmed to generate a neural feedback signal in dependence on the electronic information resource being remotely interrogated.

6. The system according to claim 1 wherein the neurological processing unit is further programmed to generate a neural feedback signal in dependence on the determined cognitive selection state.

7. The system according to claim 1 wherein the neurological processing unit is further programmed to generate at least one neural feedback signal in dependence on the electronic information resource being remotely interrogated and a resulting access state.

8. The system according to claim 7 wherein the at least one neural feedback signal comprises a plurality of perceptionally distinct neural feedback signals generated in dependence on the access state and applied to the nervous tissue in which the neurological sensor is in bioelectrical contact therewith.

9. The system according to claim 8 wherein the access state is one of; allowed and rejected.

10. The system according to claim 1 wherein the neurological processing unit is further programmed to provide bi-directional communication between the nervous tissue and the electronic information resource.

11. The system according to claim 5 further including a second neurological electronic interface operatively coupled to another neurological sensor and the electronic information resource and configured to generate the neural feedback signals in cooperation with the neurological electronic interface.

12. A method for neurologically controlling access to an electronic information resource comprising:
providing a neurological electronic interface programmed to determine whether bioelectrical signals received from a neurological sensor are indicative of a cognitive selection state and permissively allow access to an electronic information resource in dependence on the determined cognitive selection state;
providing the electronic information resource configured to operably couple to the neurological electronic interface; wherein the electronic information resource including information permissively available for remote interrogation in dependence on the determined cognitive selection state; and,
providing the neurological sensor configured to operably couple to the neurological electronic interface; wherein the neurological sensor is configured to transmit the bioelectrical signals generated by a nervous tissue in which it is to be in bioelectrical contact with the neurological electronic interface.

13. The method according to claim 12 wherein the electronic information resource is one of; an RFID device and an ISO-14443 compliant device.

14. The method according to claim 12 wherein the bioelectrical signals are generated by the nervous tissue associated with one of; a peripheral nervous system and a central nervous system when the neurological sensor is in bioelectrical contact therewith.

15. The method according to claim 12 wherein one of; the neurological electronic interface, the electronic information resource, the neurological sensor and any combination thereof is embeddable at least subcutaneously in a human host.

16. The method according to claim 12 wherein the neurological electronic interface is further programmed to generate a neural feedback signal in dependence on the electronic information resource being remotely interrogated.

17. The method according to claim 12 wherein the neurological electronic interface is further programmed to generate a neural feedback signal in dependence on the determined cognitive selection state.

18. The method according to claim 12 wherein the neurological electronic interface is further programmed to generate at least one neural feedback signal in dependence on an authentication state associated with the remote interrogation of the electronic information resource.

19. The method according to claim 18 wherein the authentication state is one of; accepted and rejected.

20. The method according to claim 12 wherein the neurological electronic interface is further programmed to provide bi-directional communication between the nervous tissue and the electronic information resource.
